(19) United States
(12) Patent Application Publication
(10) Pub. No.: US 2014/0294972 A1
(43) Pub. Date: Oct. 2, 2014

(54) Oral Suspension

(71) Applicant: Peter John Pitt White, Leicester (GB)

(72) Inventor: Peter John Pitt White, Leicester (GB)

(73) Assignee: NOVA BIO-PHARMA TECHNOLOGIES LIMITED, Leicester (GB)

(21) Appl. No.: 14/342,607

(22) PCT Filed: Sep. 7, 2012
(86) PCT No.: PCT/GB2012/052216

§ 371 Date: Jun. 18, 2014

(30) Foreign Application Priority Data
Sep. 9, 2011 (GB) 1115569.4

(51) Int. Cl. A61K 31/52

(52) U.S. Cl. CPC A61K 31/52
USPC 424/489; 514/263.3

(57) ABSTRACT

A liquid pharmaceutical composition for use in the treatment of acute lymphoblastic leukaemia (ALL) comprising 6-mercaptopurine or a salt, hydrate or solvate thereof and a pharmaceutically-acceptable excipient, wherein the composition is a suspension for oral administration, a kit of parts for the accurate dosing and administration of the liquid pharmaceutical composition, and a method for the treatment of ALL in a human patient comprising administration of a therapeutically effective amount of the liquid pharmaceutical composition.

The present invention relates to a treatment for acute lymphoblastic leukaemia (ALL). Specifically, the invention relates to pharmaceutical compositions for oral administration in the treatment of ALL, a kit of parts including the compositions and to a method for the treatment of ALL using the compositions.

### BACKGROUND OF THE INVENTION

Almost two thirds of cases of ALL occur in children aged 2 to 6 years. Peak incidence occurs in boys aged 4 years and girls aged 2 years. ALL is the most common malignancy in children, accounting for 30% of all cancers and 80% of all leukaemias.

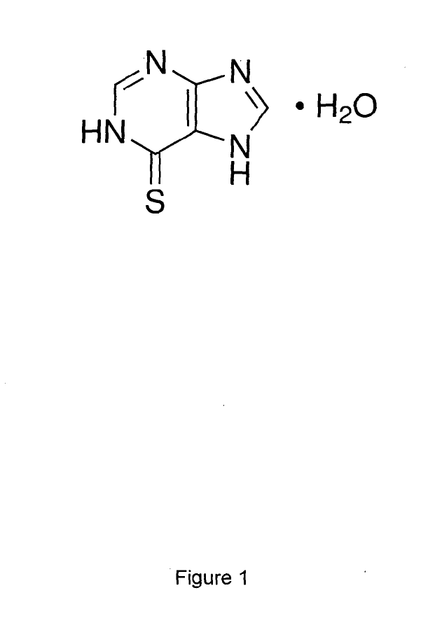

6-Mercaptopurine (6-MP), otherwise known as 3,7-dihydropurine-6-thione (shown as the monohydrate at FIG. 1), has been in clinical use for over 50 years for the treatment of ALL in adults and children as part of chemotherapy regimens. Globally, 6-MP is used in all therapy protocols for the treatment of ALL.

6-MP possesses water solubility of about 6.85 mg/mL at neutral pH.

Typical 6-MP starting doses for the treatment of ALL in children and young adults (3 months to 18 years) confirmed in a recent national randomised trial in the United Kingdom (UKALL 2003) is 75 mg/m² body surface area (BSA) for the induction, consolidation and maintenance phases. From Table 1 (below), it is clear that dependent upon the age and size of the child, 6-MP dosages vary significantly.

TABLE 1

| | | 6-MP Typical Starting Dose | 6-MP Dose |
|---|---|---|---|
| Age | BSA¹ (m²) | (per m²) | (mg) |
| 3 months | 0.27-0.33 | 75 mg | 20-25 |
| 1 year | 0.47-0.53 | 75 mg | 35-40 |
| 3 years | 0.61-0.67 | 75 mg | 46-50 |
| 5 years | 0.74-0.79 | 75 mg | 56-59 |
| 10 years | 1.07-1.13 | 75 mg | 80-85 |
| 12 years | 1.27-1.33 | 75 mg | 95-100 |
| 18 years | 1.77-1.83 | 75 mg | 133-137 |

¹Based on World Health Organisation growth charts for children.

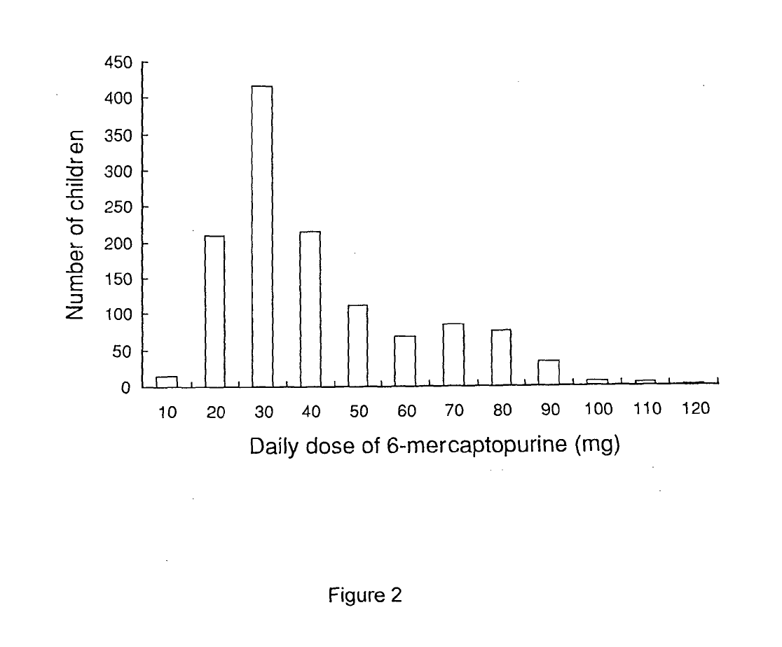

In the ALL-BFM 2000 study in Germany, the dose of 6-MP for the induction, consolidation and maintenance phases was 60 mg/m², 25 mg/m² and 50 mg/m², respectively. For children recruited into the ALL-BFM 2000 protocol the daily dose of mercaptopurine ranged from 7.5 mg to 125 mg depending on body size (FIG. 2).

Accordingly, 6-MP is considered an integral component of treatments to cure children suffering from ALL and its efficacy is unquestioned, as established over many years. Specificity of dosing and thus the need for accuracy and flexibility in dosing for efficacy is evident from the aforementioned trial data, also bearing in mind that accuracy in the administration of 6-MP is crucial for amongst other reasons the toxicity (cytotoxicity, mutagenicity, etc) of 6-MP.

There remains an enduring difficulty for patients, carers and healthcare professionals (particularly those looking after children) with the administration of 6-MP as currently there is no entirely suitable formulation available. Only a 50 mg tablet formulation of 6-MP (Puri-Nethol®, Glaxo SmithKline and Teva) has ever been licensed and marketed in the European Union. This has caused consternation amongst healthcare professionals. Additionally, within the European Medicines Agency, an age-appropriate 6-MP formulation has been identified by the Paediatric Working Party as a priority need in the chemotherapy area.

As evident from Table 1 and FIG. 2, a single or multiple 50 mg tablet(s) is/are unsuitable in delivering an accurate dose for the vast majority of patients, in particular children. This is because most patients require doses other than can be directly obtained from a 50 mg tablet in a unitary manner. In fact, less than 10% of the treated children in the trial studies received either 50 mg or 100 mg as a daily dose.

As a consequence, children being treated for ALL are often given bespoke preparations of 6-MP which raises difficulties in optimising in vivo performance. In many cases, parents and carers of children are dispensed with a 50 mg tablet form of 6-MP which is split or crushed prior to administration in attempts to attain the correct dosage.

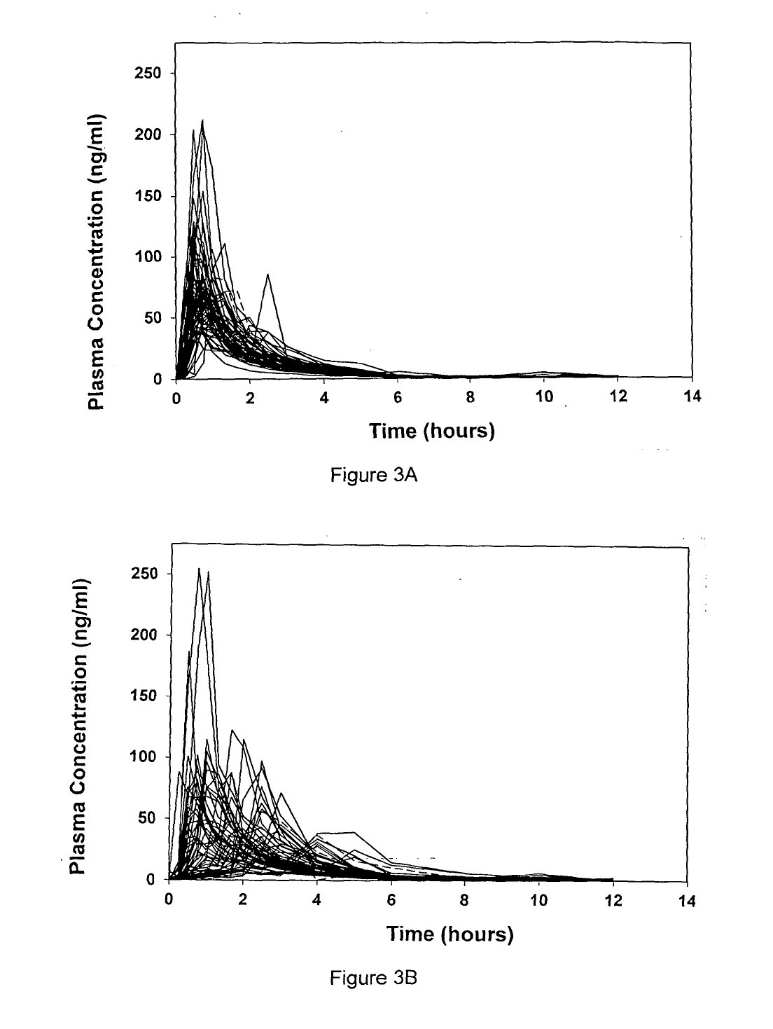

It has been demonstrated that manual splitting of 50 mg Puri-Nethol® tablets into pieces results in poor accuracy of dosing, ranging from 54% to 159% of the desired tablet mass. This was the case even if commercially-available tablet splitters were used by a pharmacist experienced in the preparation of extemporaneous formulations (see Breitkeutz J et al, Paediatric and Perinatal Drug Therapy 2007, 8(1), 31-39). Bearing in mind that the tablet form of 6-MP exhibits variability in its plasma absorption profile (see FIG. 3B), the splitting of tablets introduces additional variability in the actual dose delivered and raises significant concerns about day-to-day variations in cytotoxic effect.

It should also be noted that 6-MP is metabolised by the polymorphic enzyme thiopurinemethyl transferase (TPMT). For those patients with poor metaboliser status, it is recommended that individual doses should be reduced by a factor 10 to 15. This further underlines that splitting or crushing tablets in order to obtain an accurate starting dose is fraught with difficulties and potentially unsafe.

In summary, three major problems have been identified with the currently marketed solid dose 6-MP tableted formulations. These are a lack of accuracy and flexibility in dosing, problems of administration and compliance, and the exposure of carers (including healthcare workers and the parent carers of sick children) to cytotoxic and mutagenic dust during the manipulation of the tableted 6-MP formulation.

The present invention aims to ameliorate one or more of these problems.

### SUMMARY OF THE INVENTION

According to the present invention there is provided a liquid pharmaceutical composition for use in the treatment of acute lymphoblastic leukaemia (ALL) comprising 6-mercaptopurine or a salt, hydrate or solvate thereof, and a pharmaceutically-acceptable excipient, wherein the composition is a suspension for oral administration.

Additionally, there is provided a kit of parts comprising (a) a liquid pharmaceutical composition comprising 6-mercaptopurine and a pharmaceutically-acceptable excipient, wherein the composition is a suspension for oral administration as defined according to the invention; and (b) a plurality of syringes of different volume for the accurate dosing and administration of the liquid pharmaceutical composition.

Even further, there is provided a method for the treatment of acute lymphoblastic leukaemia in a human patient comprising administration of a therapeutically effective amount of a liquid composition comprising 6-mercaptopurine or a salt, hydrate or solvate thereof, and one or more pharmaceutically-acceptable excipients, wherein the composition is a suspension for oral administration.

The liquid composition of the present invention provides a significant improvement over the solid tableted formulation of the prior art since it provides greater flexibility and accuracy in terms of dosing, improved ease of administration and hence compliance (particularly in children), and safer handling during administration in a clinical or home environment. The compositions of the present invention have been shown to have a good storage stability profile (at ˜25° C.) of at least 28 days once exposed to the atmosphere (breaking of seal of storage container) and at least 1 year in a sealed environment.

Solid oral dosage forms such as tablets and capsules can offer advantages over liquid formulations of greater stability, improved palatability and portability. However, many children under the age of 6 years in particular have difficulty swallowing tablets or capsules (this comprises the large majority of the ALL-affected population). The efficacious liquid 6-MP compositions of the present invention help overcome these difficulties assisting greatly with patient compliance. The composition of the invention which may be administered using a syringe of pre-determined volume allows the dose of 6-MP to be tailored to patient requirements and to be delivered both accurately and safely.

Additionally, dosage accuracy is further inherent in the compositions of the invention due to their improved bioavailability over solid tableted formulations (see FIGS. 3A and 3B). This improved accuracy of dosing helps to minimise the risk of adverse reactions (dose too high) or inadequate efficacy (dose too low) resulting in safer and more efficient medication.

Furthermore, healthcare workers and other carers (particularly parents and carers of children) dispensed with the tableted form of 6-MP frequently have to resort to splitting or crushing the tablet(s) prior to administration. There are exceptional safety issues concerning the preparation of drugs for paediatric oncology. Splitting a 6-MP tablet by cutting or crushing to formulate the correct dosage requirements for a specific patient exposes both the carer and the home environment to potential cytotoxic contamination. In some circumstances where 6-MP is being administered in the home environment, there may be the risk of exposure to the unborn child of a pregnant woman in that environment.

The cytotoxic dust of 6-MP may in some cases be up to 0.46% of the total tablet mass and may be released into the surrounding environment during a tablet splitting or crushing procedure. Therefore, a danger of contamination with cytotoxic and mutagenic dust for an individual splitting or breaking the 6-MP tableted formulation without having taken protective measures (such as wearing a disposable face mask and gloves) is evident. Use of the liquid 6-MP composition of the invention overcomes this risk because each dosage can be accurately measured in a syringe based on a known concentration of the liquid composition without the need to handle a solid material.

### DETAILED DESCRIPTION OF THE INVENTION

Preferably, the liquid pharmaceutical composition for use according to the present invention comprises a suspension of 6-MP particles in a liquid. Preferably, the liquid comprises water.

The 6-mercaptopurine active agent may be present as the neutral unsolvated or unhydrated compound or as a salt, solvate or hydrate. Preferably, 6-mercaptopurine monohydrate is used in the liquid compositions of the invention.

Preferably, the particle diameter distribution of the 6-MP particles in suspension is greater than about 3 μm (D(v,0.1)) to less than about 85 μm (D(v,0.9)), with the median diameter ((D(v,0.5)) at about 35 μm to about 45 μm, and preferably 40 μm. Even more preferably, the particle diameter distribution of the 6-MP in suspension is about 25 μm (D(v,0.1)) to about 60 μm (D(v,0.9)) and most preferably is about 35 μm (D(v,0.1)) to about 45 μm (D(v,0.9)), with the median diameter ((D(v,0.5)) at about 35 μm to about 45 μm, and preferably 40 μm. The median diameter D(v,0.5) is the diameter where 50% of the distribution is above and 50% is below this value. D(v,0.9) is where 90% of the distribution is below this value. D(v,0.1) is where 10% of the distribution is below this value. Particle diameter distributions may be determined by laser diffraction methods.

The dosage amounts of 6-MP present in the liquid compositions may vary dependent upon patient needs, but preferably 6-MP is present in the liquid at about 10 to 30 mg/mL (1.0 to 3.0% w/v) and more preferably at about 15 to 25 mg/mL (1.5 to 2.5% w/v). Most preferably, the 6-mercaptopurine is present in the liquid at about 20 mg/mL.

The liquid compositions are suitable for use in any ALL patient population irrespective of age. However, preferably the compositions are for use in the paediatric treatment of ALL, most preferably in children in the 2 to 6 years age group.

The pharmaceutical excipients having application in the liquid compositions of the present invention are those readily known and available to the person skilled in the art of liquid pharmaceutical formulations. Preferably, the compositions of the invention will contain as excipients a suspending agent, a preservative, a sweetener and/or a flavouring agent, and a carrier or vehicle as the major component of the liquid phase of the compositions. A colouring agent may also be used to make a formulation more attractive to a child patient. A pH modifier such as sodium hydroxide may also be included in the compositions if necessary.

Suspending agents which may be used according to the present invention include but are not limited to xanthan gum, guar gum, polyoxyethylene sorbitol, sorbitan esters and microcrystalline cellulose. Preferably, the suspending agent is xanthan gum.

Preservatives which may be used according to the present invention include but are not limited to benzoic acid, sodium benzoate, potassium sorbate, cresol, cetrimide, citric acid and sodium citrate, and alkyl hydroxybenzoates (parabens). Preferably, the preservative is selected from an alkyl hydroxybenzoate, such as methyl hydroxybenzoate, ethyl hydroxybenzoate, propyl hydroxybenzoate (as base or sodium salt) or a combination thereof.

Sweeteners which may be used according to the present invention may be any natural or artificial sweetener. In terms of artificial sweeteners, these include but are not limited to saccharin, aspartame and sucralose. Preferably, the sweetener is aspartame. As a flavouring agent, a fruit juice concentrate is preferred, such as concentrated raspberry juice.

The carrier/vehicle used in the compositions of the invention is preferably water, although other suitable water-containing (aqueous) carriers/vehicles known to the skilled person may also be used.

In the preparation of the compositions of the invention, particulate 6-MP in the form of a powder is mixed with the excipients, preferably including a suspending agent such as xanthan gum, according to conventional techniques. Preferably, the suspensions will be formulated so as not to settle for at least several hours and preferably days, weeks or even months. However, if necessary, settled suspensions may easily be manually agitated prior to patient administration in order to re-suspend the particulate matter. The compositions of the invention are preferably stored under refrigerated conditions.

The liquid 6-MP compositions of the present invention (eg, 20 mg/mL) enable accuracy and flexibility in dosing. Once a specific dosage is known, simple dose conversion charts can be referred to so that the healthcare worker or other carer can easily establish which volume of the composition should be administered based on the concentration of the 6-MP suspension. To help enable this, in one embodiment of the kit according to the invention, packaging will contain two oral syringes: a 1 mL syringe graduated in 0.1 mL increments and a 5 mL syringe graduated in 0.2 mL increments. This particular combination of syringes permits accurate dosing for a broad range of patients undergoing treatment for ALL, in particular vulnerable children who comprise the vast majority of ALL patients.

1 mL and 5 mL syringes which may be used in accordance with the kit of the present invention are assessed for accuracy and precision, and fully comply with Ph Eur 2.9.27 guidance. A liquid composition according to the invention administered using a syringe allows the dose of 6-MP to be tailored to specific patient requirements and delivered both accurately and safely.

The kit of the invention is a multi-dose kit, which enables repeated usage during treatment. In the kit, the compositions according to the invention are held in a container(s), which may be one or more bottles, sachets, ampoules, capsules or other container suitable for storing a pharmaceutical liquid.

The present invention is now further described with reference to the Figures of the accompanying drawings as follows:

FIG. 1 is the chemical structure of 6-mercaptopurine monohydrate.

FIG. 2 is a graph of the number of children prescribed a range of daily doses of 6-mercaptopurine in the standard maintenance therapy of ALL in childhood in the ALL-BFM 2000 protocol, Germany, 2003 (Breitkeutz J et al, Paediatric and Perinatal Drug Therapy 2007, 8(1), 31-39).

FIG. 3A is a plot of the individual plasma 6-MP concentration (ng/mL) time (hours) profiles for a liquid composition according to the invention (100 mg 6-MP/5 mL) as outlined in the Examples.

FIG. 3B is a plot of the individual plasma 6-MP concentration (ng/mL) time (hours) profiles for a 50 mg Puri-Nethol 6-MP tablet as outlined in the below Example.

### Examples

A specific embodiment of the present invention is now described with reference to the following example and accompanying clinical data of FIGS. 3A and 3B. Table 2 describes a formulation according to the present invention to which the data of FIG. 3A relates.

TABLE 2
6-MP Oral Suspension

| | | | | Amount | | |
|---|---|---|---|---|---|---|
| | | Amount | | per | | |
| Component | Function | per 5 mL | | 100 mL | | % (w/v) |
| 6-Mercaptopurine | Active agent | 100 | mg | 2.0 | g | 2.0 |
| (PhEur) | | | | | | |
| Xanthan gum (PhEur) | Suspending | 25 | mg | 500 | mg | 0.5 |
| | agent | | | | | |
| Aspartame (PhEur) | Sweetener | 15 | mg | 300 | mg | 0.3 |
| Concentrated raspberry | Natural | 0.25 | mL | 5.0 | mL | 5.0 |
| juice (BP 1988) | flavouring | | | | | |
| Methyl hydroxybenzoate | Preservative | 5.0 | mg | 100 | mg | 0.10 |
| (PhEur) | | | | | | |
| Propyl hydroxybenzoate | Preservative | 0.75 | mg | 15 | mg | 0.015 |
| (PhEur) | | | | | | |
| Water | Carrier/ | 5.0 | mL | 100 | mL | To 100 |
| | Vehicle | | | | | |

The particulate active pharmaceutical ingredient 6-MP (particle diameter distribution of greater than about 3 μm (D(v,0.1)) to less than about 85 μm (D(v,0.9)), with median diameter (D(v,0.5)) at 40 μm) was obtained from Fermion (Finland), xanthan gum was obtained from CPKelco (Atlanta, Ga., USA), and aspartame, concentrated raspberry juice, methyl hydroxybenzoate and propyl hydroxybenzoate were obtained from Fagron (Netherlands).

Particle diameter distribution was determined by laser diffraction methods on a Malvern Mastersizer S particle size analyzer (software version 3.00) manufactured by Malvern Instruments Ltd (United Kingdom). Average D(v,0.1) μm, D(v,0.5) μm and D(v,0.9) μm values were recorded after four particle count scans.

A single, comparative bioavailability study was conducted involving a liquid 6-MP suspension (100 mg/5 mL) according to the present invention. The study, a single-dose, randomised, crossover design, was conducted in 60 fasted, healthy male volunteers. After an overnight fast, subjects were dosed with either one 50 mg Puri-Nethol tablet (reference) or 2.5 mL of the mercaptopurine oral suspension 100 mg/5 mL (test) in accordance with the suspension as outlined in Table 2.

An assessment of bioequivalence showed that that the exemplified 6-MP composition has bioequivalence to a tablet form of 6-MP, with respect to AUC, but not Cₘₐₓ. The mean ratio and 90% CI for AUC (114% and 108-121% respectively) easily lie in the target 80-125% range accepted for bioequivalence, whereas mean and 90% confidence intervals for Cₘₐₓ (139% and 122-158% respectively) lie outside these criteria.

While bioequivalence criteria for Cₘₐₓ are not met, the plasma concentration profiles clearly indicate that the liquid composition according to the invention performs more consistently and predictably than the tablet. The individual plasma versus concentration time profiles for the liquid composition suggest moderately rapid absorption and are on the whole well defined, sharp profiles (FIG. 3A). In contrast, the tablet profiles display inconsistent, erratic absorption with significant lag phase in many cases suggestive of dissolution rate limited absorption (FIG. 3B). This is mathematically demonstrated by a substantially lower between-subject variability in Cₘₐₓ (%CV, 46% vs 69%) and narrower Cₘₐₓ range (37.7-212 vs 6.7-255 ng/mL) for the suspension compared to the tablet.

Overall, liquid composition was well-tolerated in the study subjects and no adverse events were observed.

The greater variability in the absorption of 6-MP following tablet administration compared to administration of the liquid composition of the invention demonstrates an unexpected benefit in terms of improved accuracy of the 6-MP dosage when using the compositions of the invention.

Table 3 provides a comparative benefit summary of the liquid 6-MP compositions according to the invention compared to the known tableted formulation.

TABLE 3

| | Benefit of liquid |
|---|---|
| | 6-mercaptopurine |
| | composition of invention in |
| Potential Benefit | childhood ALL |
| Reduced adverse drug reactions or | Yes, decreased risk of |
| reduced potential for medication errors? | medication errors. |
| Improved dosing scheme or method of | Yes, the oral suspension |
| administration? | formulation improves ease |
| | of administration |
| | and compliance. |
| Improved safety for carers currently | Yes. |
| splitting mercaptopurine tablets? | |
| Availability of new clinically relevant | Yes. |
| age-appropriate formulation? | |
| Different mechanism of action leading to | More accurate and |
| improved safety and efficacy? | consistent dosing possible. |

A further composition in accordance with the present invention is detailed at Table 4 as follows.

TABLE 4

| | | Formula (mg unless | | | |
|---|---|---|---|---|---|
| | | specified) | | | |
| | | Per 1 ml | Per 5 ml | | Quality |
| Component | Function | dose | dose | Per 100 ml | standard |
| Mercaptopurine¹ | Active | 20.0 | 100.0 | 2000.0 | Ph. Eur |
| Xanthan Gum | Viscosity | 5 | 25 | 500 | Ph. Eur |
| | modifier | | | | |
| Aspartame | Sweetener | 3.0 | 15.0 | 300 | Ph. Eur |
| Concentrated | Flavouring | 0.05 mL | 0.25 mL | 5 mL | BP 1988 |
| Raspberry | Agent | | | | |
| Juice.² | | | | | |
| Juice of the | | | | | |
| raspberry | | | | | |
| Rubus idaeus L. | | | | | |
| Sucrose added to | | | | | |
| adjust weight per | | | | | |
| mL in the final | | | | | |
| concentrate to | | | | | |
| 1.30 to 1.36 g. | | | | | |
| Methyl para- | Antimicrobial | 1.145³ | 5.725 | 114.5 | Ph. Eur |
| hydroxybenzoate, | preservative | | | | |
| sodium. | | | | | |
| Ethyl para- | Antimicrobial | 0.566⁴ | 2.83 | 56.6 | Ph. Eur |
| hydroxybenzoate, | preservative | | | | |
| sodium. | | | | | |
| Potassium | Antifungal | 1.0 | 5 | 100 | Ph. Eur |
| sorbate. | preservative | | | | |
| Sodium hydroxide. | pH modifier | qs | qs | qs | Ph. Eur |
| Water.⁵ | Diluent/vehicle | To 1 mL | To 5 mL | To 100 mL | Ph. Eur |

¹The European Pharmacopoeia defines mercaptopurine as mercaptopurine monohydrate. The formulation contains 20 mg mercaptopurine monohydrate (20 mg mercaptopurine Ph. Eur.) per ml.
²Sulphur dioxide is present as a preservative ≦4700 ppm).
³Equivalent to 1.0 mg/mL of methyl hydroxybenzoate base.
⁴Equivalent to 0.5 mg/mL of ethyl hydroxybenzoate base.
⁵Complies with the Ph. Eur. monograph for purified water or water for injections in bulk.

The suspension is filled into amber glass bottles at a nominal fill of 100 mL, and closed with a polyethylene screw cap. In use, the screw cap is replaced with a polyethylene insert to facilitate use of the dosing syringe. Graduated 1 mL and 5 mL dosing syringes are provided with each bottle.

1. A liquid pharmaceutical composition for use in the treatment of acute lymphoblastic leukaemia, the composition comprising 6-mercaptopurine a salt, a hydrate or a solvate thereof, and a pharmaceutically-acceptable excipient, wherein the composition is a suspension for oral administration.

2. The liquid pharmaceutical composition for use according to claim 1, wherein the suspension comprises the 6-mercaptopurine, salt, hydrate or a solvate thereof as particles suspended in a liquid.

3. The liquid pharmaceutical composition of claim 2, wherein the liquid comprises water.

4. The liquid pharmaceutical composition of claim 3, wherein the 6-mercaptopurine, salt, hydrate or a solvate thereof is 6-mercaptopurine monohydrate.

5. The liquid pharmaceutical composition of claim 3, wherein the 6-mercaptopurine, salt, hydrate or a solvate thereof is present in the liquid at about 10 to 30 mg/mL.

6. The liquid pharmaceutical composition of claim 5, wherein the 6-mercaptopurine, salt, hydrate or a solvate thereof is present in the liquid at about 15 to 25 mg/mL.

7. The liquid pharmaceutical composition of claim 6, wherein the 6-mercaptopurine, salt, hydrate or a solvate thereof is present in the liquid at about 20 mg/mL.

8. (canceled)

9. The liquid pharmaceutical composition of claim 1, wherein the pediatric use is for children aged about 2 to about 6 years.

10. The liquid pharmaceutical composition of claim 5, wherein the diameter distribution of the particles of 6-mercaptopurine, salt, hydrate or a solvate thereof in suspension is greater than about 3 μm (D(v,0.1)) to less than about 85 μm(D(v,0.9)), with median diameter (D(v,0.5)) at 40 μm.

11. The liquid pharmaceutical composition of claim 10, wherein the diameter distribution of the particles of 6-mercaptopurine, salt, hydrate or a solvate thereof is about 25 pm (D(v,0.1)) to about 60 μιτι (D(v,0.9)).

12. The liquid pharmaceutical composition for use according to claim 11, wherein the diameter distribution of the particles of 6-mercaptopurine, salt, hydrate or a solvate thereof is about 35 μιτι (D(v,0.1)) to about 45 μιτι (D(v,0.9)).

13-15. (canceled)

16. A method for the treatment of acute lymphoblastic leukaemia in a human patient in need thereof, the method comprising a therapeutically effective amount of a liquid composition comprising 6-mercaptopurine a salt, a hydrate or a solvate thereof, and one or more pharmaceutically-acceptable excipients, wherein the composition is administered orally as a suspension.

17. The method according to claim 16, wherein the suspension comprises particles of 6-mercaptopurine, salt, hydrate or a solvate thereof suspended in a liquid.

18. The method of claim 17, wherein the liquid comprises water.

19. The method of claim 18, wherein the 6-mercaptopurine, salt, hydrate or a solvate thereof is 6-mercaptopurine monohydrate.

20. The method of claim 18, wherein the 6-mercaptopurine, salt, hydrate or a solvate thereof is present in the liquid at about 10 to 30 mg/mL.

21. (canceled)

22. The method of claim 16, wherein the use is for children aged 2 to 6 years.

23. The method of claim 20, wherein wherein the diameter distribution of the particles of 6-mercaptopurine, salt, hydrate or a solvate thereof in suspension is greater than about 3 μm (D(v,0.1)) to less than about 85 μm (D(v,0.9)), with median diameter (D(v,0.5)) at 40 μm.

24. The method of claim 23, wherein the diameter distribution of the particles of 6-mercaptopurine, salt, hydrate or a solvate thereof is about 25 μm (D(v,0.1)) to about 60 μm (D(v,0.9)).

25. The method of claim 24, wherein the diameter distribution of the particles of 6-mercaptopurine, salt, hydrate or a solvate thereof is about 35 μm (D(v,0.1)) to about 45 μm (D(v,0.9)).
